(19) United States
(12) Patent Application Publication
(10) Pub. No.: US 2024/0087719 A1
(43) Pub. Date: Mar. 14, 2024

(54) METHODS AND SYSTEMS FOR CALCULATING AN EDIBLE SCORE IN A DISPLAY INTERFACE

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 18/377,135

(22) Filed: Oct. 5, 2023

Related U.S. Application Data
(63) Continuation-in-part of application No. 18/195,143, filed on May 9, 2023.
Application No. 18/195,143 is a continuation-in-part of application No. 17/007,227, filed on Aug. 31, 2020.
Application No. 17/007,227 is a continuation-in-part of application No. 16/983,034, filed on Aug. 3, 2020, now Pat. No. 11,688,506.

(51) Int. Cl. G16H 20/60

(52) U.S. Cl. CPC G16H 20/60

(57) ABSTRACT

A system for calculating a score for an edible in a display interface, the system having a computing device configured to determine an edible of interest relating to a user, receive nourishment information relating to the edible of interest to the user, wherein the nourishment information includes a plurality of ingredients, calculate one or more nutrient biodiversity scores as a function of the nourishment information including evaluating each ingredient of the plurality of ingredients, wherein evaluating each ingredient includes, extracting at least a nutrient from each ingredient of the plurality of ingredients and calculating a nutrient biodiversity score for the at least a nutrient, determine a nutritional requirement as a function of at least the nourishment information, and display the nutritional requirement and the one or more nutrient biodiversity scores of the edible of interest through a display interface.

### CROSS-REFERENCE TO RELATED APPLICATIONS

This application is a continuation-in-part of Non-provisional application Ser. No. 18/195,143, filed on May 9, 2023, and entitled “METHODS AND SYSTEMS FOR CALCULATING AN EDIBLE SCORE IN A DISPLAY INTERFACE,” which is a continuation-in-part of Non-provisional application Ser. No. 17/007,227 filed on Aug. 31, 2020 and entitled “METHODS AND SYSTEMS FOR CALCULATING AN EDIBLE SCORE IN A DISPLAY INTERFACE,” which is a continuation-in-part of Non-provisional application Ser. No. 16/983,034 filed on Aug. 3, 2020 and entitled “METHODS AND SYSTEMS FOR CALCULATING AN EDIBLE SCORE IN A DISPLAY INTERFACE,” each of which are entirely incorporated herein by reference.

### FIELD OF THE INVENTION

The present invention generally relates to the field of nourishment. In particular, the present invention is directed to methods and systems for calculating an edible score in a display interface.

### BACKGROUND

Understanding how much to eat, and what types of foods to eat can be challenging. Frequently, users are overloaded with conflicting information. A void exists to deliver custom food suggestions that indicate how a particular food will uniquely affect a consumer.

### SUMMARY OF THE DISCLOSURE

In an aspect, a system for calculating a score for an edible in a display interface is described. the system includes a computing device configured to determine an edible of interest relating to a user, receive nourishment information relating to the edible of interest to the user, wherein the nourishment information includes a plurality of ingredients, calculate one or more nutrient biodiversity scores as a function of the nourishment information including evaluating each ingredient of the plurality of ingredients, wherein evaluating each ingredient includes, extracting at least a nutrient from each ingredient of the plurality of ingredients and calculating a nutrient biodiversity score for the at least a nutrient, determine a nutritional requirement as a function of at least the nourishment information, and display the nutritional requirement and the one or more nutrient biodiversity scores of the edible of interest through a display interface.

In another aspect, a method for calculating a score for an edible in a display interface is described. The method includes determining, using a computing device, an edible of interest relating to a user, receiving, using the computing device, nourishment information relating to the edible of interest to the user, wherein the nourishment information comprises a plurality of ingredients, calculating, using the computing device, one or more nutrient biodiversity scores as a function of the nourishment information including evaluating each ingredient of the plurality of ingredients, wherein evaluating each ingredient includes extracting at least a nutrient from each ingredient of the plurality of ingredients and calculating a nutrient biodiversity score for the at least a nutrient, determining, using the computing device, a nutritional requirement as a function of at least the nourishment information and displaying, using the computing device, the nutritional requirement and the one or more nutrient biodiversity scores of the edible of interest through a display interface.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for calculating an edible score in a display interface;

FIG. 2 is a diagrammatic representation of a performance profile;

FIG. 3 is a block diagram illustrating an exemplary embodiment of a user database;

FIG. 4 is a block diagram illustrating an exemplary embodiment of an edible database;

FIG. 5 is a block diagram illustrating an exemplary embodiment of a machine-learning module;

FIGS. 6A-6C are diagrammatic representations of display interface;

FIG. 7 is a process flow diagram illustrating an exemplary embodiment of a method of calculating an edible score in a display interface;

FIG. 8 is a process flow diagram illustrating an exemplary embodiment of a method of calculating an edible score in a display interface

FIG. 9 is yet another process flow diagram illustrating an exemplary embodiment of a method of calculating an edible score in a display interface; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for calculating an edible score in a display interface. In an embodiment, a performance profile is used together with an edible of interest, and using a score machine-learning process, to calculate an edible score. An edible score is displayed within display interface, to aid a user in making informed food selections.

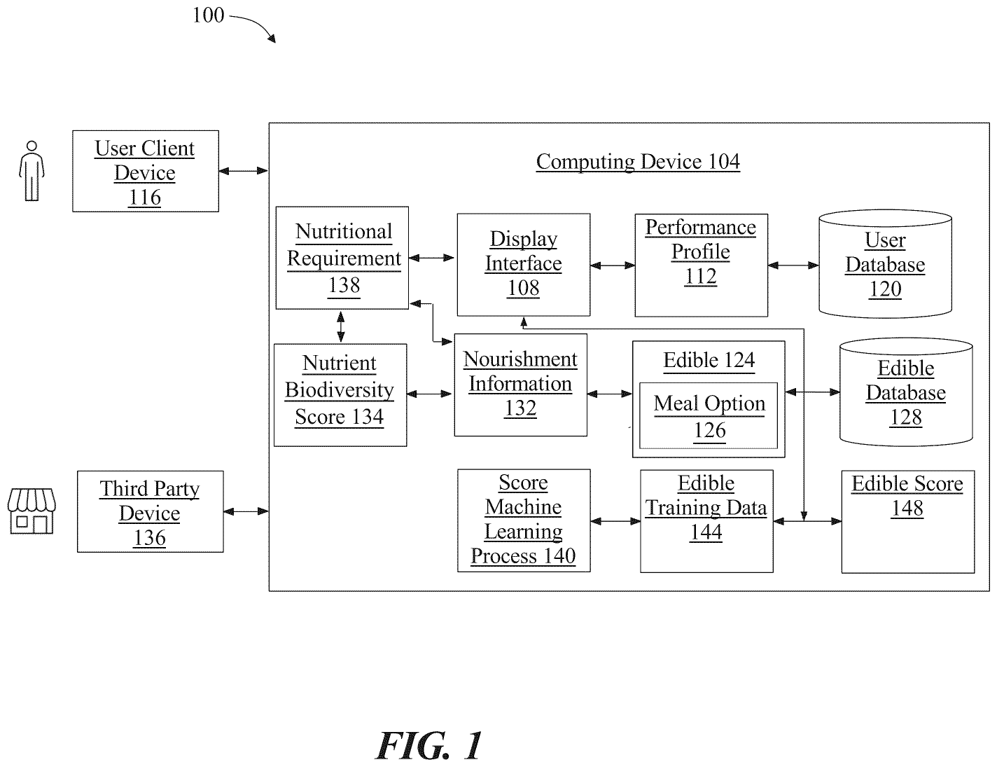

Referring now to the drawings, FIG. 1 illustrates an exemplary embodiment 100 of a system for calculating an edible score in a display interface. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or connect with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or connect with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an association, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be transmitted to and/or from a computer and/or a computing device 104. Computing device 104 may include but is not limited to, for example, a computing device 104 or cluster of computing devices 104 in a first position and a second computing device 104 or cluster of computing devices 104 in a second position. Computing device 104 may include one or more computing devices 104 dedicated to data storage, security, dispersal of traffic for load balancing, and the like. Computing device 104 may distribute one or more computing tasks as described below across a plurality of computing devices 104 of computing device 104, which may operate in parallel, in series, redundantly, or in any other manner used for dispersal of tasks or memory between computing devices 104. Computing device 104 may be implemented using a “shared nothing” architecture in which data is cached at the operative, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence recurrently until a desired or commanded outcome is achieved; repetition of a step or a sequence of steps may be performed iteratively and/or recursively using outputs of previous repetitions as inputs to subsequent repetitions, assembling inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to initiate a display interface within computing device 104. A “display interface,” as used in this disclosure, is a user interface that allows a user to interface with computing device 104 through graphical icons, audio indicators, command labels, text navigation and the like. Display interface 108 may include a form or other graphical element having display fields, where one or more elements of information may be displayed. Display interface 108 may include slides or other user commands that may allow a user to select one or more characters. Display interface 108 may include free form textual entries, where a user may type in responses and/or messages. Display interface 108 may display data output fields including text, images, or the like. Display interface 108 may include data input fields such as text entry windows, drop-down lists, buttons, checkboxes, radio buttons, sliders, links, or any other data input interface that may capture user interaction as may occur to persons skilled in the art upon reviewing the entirety of this disclosure. Display interface 108 may be provided, without limitation, using a web browser, a native application, a mobile application, or the like.

With continued reference to FIG. 1, computing device 104 is configured to retrieve a performance profile 112 relating to a user. A “performance profile,” as used in this disclosure, is a compilation of one or more elements of data relating to one or more aspects of a user's body, lifestyle, beliefs, and/or practices. An element of data may include biological extraction data. A “biological extraction,” as used in this disclosure, is any data indicative of a person's physiological state; physiological state physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. A biological extraction includes without limitation any biological extraction as described in U.S. Nonprovisional application Ser. No. 16/530,329 filed on Aug. 2, 2019 and entitled “METHODS AND SYSTEMS FOR GENERATING COMPATIBLE SUBSTANCE INSTRUCTION SETS USING ARTIFICIAL INTELLIGENCE,” the entirety of which is incorporated herein by reference. An element of data may include any information relating to current levels of age related degradation. Age related degradation may include any physiological changes that occur within the human body. For example, age related degradation may include thinning and loss of elasticity of the skin. In yet another non-limiting example, age related degradation may include a decrease in the production of natural oils, which may cause the skin to become drier. An element of data may include information relating to a user's sleep patterns, including information describing quantities of sleep, quality of sleep, information pertaining to sleep cycles, nighttime wakefulness, daytime drowsiness, daytime sleeping patterns, and the like. For example, an element of data may specify that a user sleeps on average over the previous one week, a total of 7 hours each night. An element of data may include information relating to a user's fitness patterns such as how much exercise a user engages in, types of exercise that a user engages in, frequency of exercise, exercise groups that a user is a member of, exercise classes that a user partakes in, and the like. For example, an element of data may specify that over the previous week, a user exercised a total of one hundred twenty minutes, of which forty minutes were spent on weight bearing exercise, and eighty minutes were spent on cardiovascular exercise that included a mix of walking and running. An element of data may include information relating to a user's nutritional status, such as any information describing any self-reported food allergies, food sensitivities, dietary requests, style of eating, and the like that the user may be following. For example, an element of data may specify that a user follows a vegan diet for ethical and personal religious reasons. A performance profile may include any information suitable for use as a nourishment score, as described in U.S. Nonprovisional application Ser. No. 16/886,673 filed on May 22, 2020, and entitled “METHODS AND SYSTEMS FOR GEOGRAPHICALLY TRACKING NOURISHMENT SELECTION,” the entirety of which is incorporated herein by reference.

With continued reference to FIG. 1, an element of data may include information relating to a user's stress levels, such as information describing how often a user feels stressed in an average week, how much stress on average a user feels over a specified period of time, triggers of stress for the user, stress coping mechanisms, and the like. For example, an element of data may specify that a user feels extremely stressed out before presentations, but that deep breathing exercises help mitigate feelings of stress for the user. In yet another non-limiting example, an element of data may specify that a user feels most stressed out at the beginning of the week when the user has a lot of items to complete, and the user feels less stressed out as the week progresses, and the user starts to complete certain items. An element of data may include information relating to a user's toxicity level. A toxicity level may include any information describing a degree to which a substance and/or any mixture of one or more substances has damaged a user's body. A toxicity level may contain one or more indicators of substances that include, but are not limited to heavy metals, solvents, volatile organic compounds, pesticides, bisphenol A, phthalates, parabens, electromagnetic field radiation, heterocyclic amines, intestinal bacteria, yeast, candida, infectious disease, food additives, chemicals, glyphosate, insulin resistance, medications, stress, and/or emotions. For example, a toxicity level may include one or more measurements of heavy metals such as aluminum, antimony, arsenic, barium, beryllium, bismuth, cadmium, cesium, gadolinium, lead, mercury, nickel, palladium, platinum, tellurium, thallium, thorium, tin, tungsten, uranium, and the like. An element of data may include information relating to a user's emotional and/or psychological state, including one or more indicators of age, sex, financial well-being, sedentary lifestyle, career stress, personal relationships, significant life events such as a death in the family or a divorce, unresolved emotional trauma, post-traumatic stress disorder, and the like.

With continued reference to FIG. 1, a performance profile 112 may be obtained utilizing a questionnaire. A “questionnaire,” as used in this disclosure, is an instrument containing one or more prompts for information from a participant such as a user. A questionnaire may include one or more questions prompting a user to respond to a request to obtain information relating to a performance profile 112. In an embodiment, computing device 104 may display a questionnaire within display interface 108. A questionnaire may include one or more question styles and/or types of questions including but not limited to true or false questions, multiple choice questions, ordering questions, open ended essay questions, fill in the blank questions, matching questions, and the like. For example, a questionnaire may include a question asking a user to describe the user's sleeping habits over the course of the previous night. One or more answers to a questionnaire may be obtained from a user client device 116, operated by a user. A user client device 116 may include without limitation, an additional computing device such as a mobile device, laptop, desktop computer, and the like. A user client device 116 may include, without limitation, a display in communication with computing device 104.

With continued reference to FIG. 1, a performance profile 112 may be obtained from sensor data. Sensor data may be obtained from any sensor and/or medical device configured to capture sensor data concerning a user, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmography equipment, or the like. A sensor may include any electromagnetic sensor, including without limitation electroencephalographic sensors, magnetoencephalographic sensors, electrocardiographic sensors, electromyographic sensors, or the like. A sensor may include a temperature sensor. A sensor may include any sensor that may be included in a mobile device and/or wearable device, including without limitation a motion sensor such as an inertial measurement unit (IMU), one or more accelerometers, one or more gyroscopes, one or more magnetometers, or the like. A wearable and/or mobile device sensor may capture step, gait, and/or other mobility data, as well as data describing activity levels and/or physical fitness. A wearable and/or mobile device sensor may detect heart rate or the like. A sensor may detect any hematological parameter including blood oxygen level, pulse rate, heart rate, pulse rhythm, blood sugar, and/or blood pressure. A sensor may be configured to detect internal and/or external biomarkers and/or readings. A sensor may be a part of system 100 or may be a separate device in communication with system 100.

Still referring to FIG. 1, performance profile 112 may include a logged user performance metric. A “logged user performance metric,” as used in this disclosure, is a description of any previously consumed edible. A logged user performance metric may include a description of a meal that a user ate, a series of meals, and the like. One or more logged user performance metrics may be stored within user database 120. A logged user performance metric may contain a timestamp, indicating the date and time when a user consumed a logged user performance metric. A logged user performance metric may include other information about a meal, such as any methods of preparing the meal, any ways in which the meal was customized to the user's preferences, how well the user liked or disliked the meal, and/or what serving size of the meal the user consumed.

With continued reference to FIG. 1, information relating to a performance profile 112 may be stored within user database 120. User database 120 may be implemented, without limitation, as a relational database, a key-value retrieval datastore such as a NOSQL database, or any other format or structure for use as a datastore that a person skilled in the art would recognize as suitable upon review of the entirety of this disclosure.

With continued reference to FIG. 1, computing device 104 is configured to determine an edible of interest. An “edible,” as used in this disclosure, is any substance consumed by a human being. Edible 124 may include a single ingredient, a combination of one or more ingredients, a meal including breakfast, lunch, dinner, snack, dessert, beverage, and/or any combination thereof. For instance and without limitation, edible 124 may include a breakfast option such as buckwheat pancakes topped with fresh berries and raw honey. In yet another non-limiting example, edible 124 may include a beverage such as ginger lime kombucha. An “edible of interest,” as used in this disclosure, is any edible 124 that computing device 104 selects to present to a user within display interface 108, as a possible item that a user may be interested in and/or may wish to consume. An edible of interest may be received from user client device 116. In one or more embodiments, edible of interest may be received through one or more food delivery software on user client device. For example, and without limitation, a user may select a food item on a food delivery software to consume wherein computing device 104 may receive the selected food item. In one or more embodiments, edible of interest may include food items that the user wishes to consume. These food items may include homemade foods, food ordered from local restaurants and the like. In one or more embodiments, computing device 104 may be communicatively connected to user client device 116 wherein computing device 104 may receive edible of interest that the user seeks to ingest. In one or more embodiments, computing device 104 may receive edible of interest using application program interfaces (API) or by monitoring user emails or communications. In one or more embodiments, computing device may locate receipts within emails and/or communications to determine purchased food items which may be considered an edible of interest. In one or more embodiments, computing device 104 may use one or more APIs to receive food items from food delivery apps, grocery apps, meal apps and the like. In one or more embodiments, a user may select an edible 124 to consume and transmit the selected edible to computing device 104. An edible of interest may be identified based on information contained within user database 120, including information relating to a user's dietary habits. A “dietary habit,” as used in this disclosure, is data including any character, numerical, and/or symbolic data representing a user's eating patterns. A dietary habit may include information relating to a user's food preferences, style of eating, food likes, food dislikes, mealtimes, average number of meals consumed each day, and the like. For instance and without limitation, a dietary habit may specify that a user consumes two meals per day, with a first meal generally around 1 PM, and a second meal around 6 PM. In yet another non-limiting example, a dietary habit may specify that a user follows a vegan diet for breakfast and lunch but consumes seafood at dinner. In yet another non-limiting example, a dietary habit may specify that a user dislikes asparagus, and the user abstains from eating asparagus. Information relating to a use's dietary habits may be stored within user database 120. In one or more embodiments, dietary habits may include ingredients, foods and such that the user likes or dislikes. In one or more embodiments, dietary habits may further include foods in which the user may be allergic to or is just generally refraining from eating.

With continued reference to FIG. 1, edible of interest may include a meal option 126. A “meal option,” as used in this disclosure, is a proposed meal item, including any meal and/or portion of a meal such as but not limited to any breakfast, lunch, dinner, snack, and/or beverage. In one or more embodiments, meal option 126 may include a suggestion for a future meal such as meal for the following morning. In one or more embodiments, edible of interest may include more than one edibles that a user may consume wherein each meal option 126 is associated with a singular edible. In one or more embodiments, meal option 126 may include a suggest meal for the user. In one or more embodiments, edible of interest may include one or more meal options 126 wherein the one or more meal options 126 may be used to generate a meal plan for the user. For example, and without limitation, one or more meal options 126 may include a weekly or monthly meal plan, wherein each meal option 126 may be associated with a particular meal during the week. Information pertaining to a meal option 126 may be stored within a database, such as user database 120 or edible database 128. For example, edible database 128 may include information listing a plurality of edibles 124 or edibles of interest wherein each edible or edible of interest may be selected as a meal options. In one or more embodiments, each edible 124 or edible of interest on database may be selected and used as a particular meal option for a particular time of day or week. For example, and without limitation, edible database 128 may include one or more edibles wherein each edible may be used for a particular meal options. In one or more embodiments, edible database 128 may contain a list of classified edibles based to one or more meal types. For example, edibles 124 may be classified to a breakfast, lunch, dinner and/or snack grouping. In one or more embodiments, computing device 104 may select one or more meal options based on each categorization of meal type. For example, a meal option 126 for breakfast may be selected from a categorization of foods classified as breakfast, whereas a meal option 126 for lunch may be selected from a categorization of foods categorized as lunch. In one or more embodiments, computing device 104 may select meal options 126 based on their meal type categorization. For example and without limitation, one or more meal options 126 that are available for breakfast, such as a first meal option consisting of buckwheat pancakes with maple syrup, a second meal option consisting of a yogurt parfait with granola and fresh berries, and a third meal option consisting of scrambled eggs with toast and bacon. Computing device 104 may also select a meal option 126 from a list containing a plurality of meal options 126 within a specified geographical area. Meal options 126 stored within edible database 128 may be organized into one or more lists based on meal options 126 that are available within a geographical area. A geographical area may include a global position system (GPS) of a location, including for example, a GPS location of a remote device 116. A geographical area may include a description of the latitude and longitude of a position where a remote device 116 is currently located and/or may be located in the future. A geographical area may be identified using one or more inputs received from remote device 116. For example, computing device 104 may receive a textual input from remote device 116 that specifies a user is located in San Francisco, California. In such an instance, computing device 104 identifies meal options 126 available in San Francisco, from a list indicating which meal options 126 are currently available within San Francisco. Information relating to meal options 126 that are available within a geographical area may be contained within one or more lists stored within user database 120. A meal option 126, includes a portion size, indicating what size and/or quantities of a meal option are available. For example, a portion size may indicate that a meal containing chicken alfredo is available in small, medium, large, and extra-large portions. Information relating to available meal options 126 and available portion sizes may be displayed within display interface 108. In one or more embodiments, meal options may be generated using a machine learning process such as any machine learning process as described in this disclosure. In one or more embodiments, the machine learning process may receive an input such user dietary habits (as described below) and an output may include one or more meal options. In one or more embodiments, training data may include a plurality of user dietary habits correlated to a plurality of meal options. In one or more embodiments, computing device 104 may be configured to determine a plurality of edible of interests wherein each edible of interest may include a meal option for a particular time frame. In one or more embodiments, computing device 104 may generate a singular edible of interest and/or an edible of interest having a plurality of meal options wherein each meal option includes an edible of interest for a particular time of day, week and the like.

With continued reference to FIG. 1, computing device may be configured to determine edible of interest and/or one or more meal options through a support advisor. “Support advisor” for the purposes of this disclosure is an individual who holds themselves out as having the requisite knowledge to assist in the selection of edibles of interest or meal options. For example, and without limitation, support advisor may include a nutritional specialist, a doctor, a fitness trainer and the like. In one or more embodiments, a database such as user database and/or edible database may contain a plurality of support advisors. In one or more embodiments, computing device 104 may be configured to select a support advisor from the plurality of support advisors. Selection may include selecting a support advisor that is currently available, selecting a support advisor within the same geographic region as the user as indicated by performance profile and/or any other user-related information and the like. In one or more embodiments, a support advisor may be selected based on a user's performance profile 112. For example, and without limitation, a particular support advisor may be selected based on deficiencies within the performance profile 112. Continuing, a support advisor proficient in weight loss may be selected for a user with a performance profile indicating the user is overweight. In one or more embodiments, support advisor may be selected based on biological extraction and/or performance profile wherein each user may be classified to a support advisor using as classification algorithm such as any classification algorithm as described in this disclosure. In one or more embodiments, the selected support advisor may receive performance profile 112, biological extraction and/or logged user performance metrics and generate one or more meal options as a function of the performance profile 112, biological extraction and/or logged user performance metrics. In one or more embodiments, the support advisor may generate one or more meal options and/or edibles of interest and transmit them to computing device.

With continued reference to FIG. 1, computing device 104 may determine an edible of interest based on previous food items consumed by the user. For example, and without limitation, a logged user performance metric and/or performance profile 112 may include other information about a meal, such as any methods of preparing the meal, any ways in which the meal was customized to the user's preferences, how well the user liked or disliked the meal, and/or what serving size of the meal the user consumed. Continuing, computing device 104 may determine an edible of interest based on previous foods in which the user liked or disliked. In one or more embodiments, following each iteration, a user may be configured to input information associated with the edible of interest consumed wherein computing device 104 may store the information on user database 120 and use the information for future iterations. In one or more embodiments, performance profile may include a plurality of logged user performance metrics indicating various edibles of interest and the effect they had on the user. In one or more embodiments, logged user performance metrics may include ratings on how much the user preferred the edible 124 wherein computing device may be configured to select edibles 124 with the highest ratings. In one or more embodiments, logged user performance metrics may further include various physiological effects the food items had on the user wherein computing device 104 may be configured to determine an edible of interest based on the physiological effects. For example, and without limitation, logged user performance metrics may indicate that a user had a positive physical reaction to a particular food item wherein computing device may select the particular food item for future iterations. Similarly, a user may indicate that they had a negative reaction to a particular food item wherein computing device 104 may not select the edible of interest for future iterations.

With continued reference to FIG. 1, computing device 104 may be configured to retrieve a plurality of performance profiles associated with a plurality of individuals. In one or more embodiments, the plurality of performance profiles may be retrieved from a database such as user database and/or any other database as described in this disclosure. In one or more embodiments, the plurality of individuals may include individuals who are associated with one another such as but not limited to, family members, friends, individuals living within the same house, individuals who share meals together and the like. In one or more embodiments, computing device 104 may receive a plurality of performance profiles and determine an edible of interest that is compatible with the plurality of performance profiles. In one or more embodiments, each performance profile may contain a plurality of logged performance metrics wherein each set of the plurality of logged performance metrics include plurality of logged user performance metrics for each performance profile. In one or more embodiments, computing device 104 may select an edible of interest that does not contain a negative response as indicated within the plurality of performance profiles and/or the plurality of logged performance metrics. In one or more embodiments, computing device 104 may select an edible of interest having the largest positive response as indicated by the plurality of logged performance metrics. In one or more embodiments, a logged user performance metric may contain a logged performance metric pertaining to the user whereas a logged performance metric may include a logged user performance metric pertaining to any individual including user. In one or more embodiments, plurality of performance profiles may include a plurality of logged performance metrics indicating a plurality of individual's reactions to various food items. In one or more embodiments, a user may select through user client device 116 one or more performance profiles in which they associate themselves with wherein computing device 104 may use said performance profiles to generate and/or determine edible of interest. In one or more embodiments, computing device 104 may generate a common edible of interest wherein each individual associated with the plurality of performance profiles may be able to consume the edible of interest.

With continued reference to FIG. 1, computing device 104 may identify an edible of interest using information relating to a user's geolocation. A “geolocation,” as used in this disclosure, is a real-world geographical location of a user. A geolocation may include a global positioning system (GPS) of a user, and/or geographic coordinates that specify the latitude and longitude of particular location where a user is located. A geolocation may be obtained from a radar source, user client device 116, self-reported by the user, and the like. Computing device 104 receives an element of user geolocation data and identifies a plurality of edibles as a function of the element of user geolocation data. Information pertaining to edible 124 may be stored within edible database 128. Edible database 128 may be implemented as any data structure suitable for use as user database 120, as described above in more detail. In an embodiment, computing device 104 may generate a query, to search for edibles 124 that may be available within the user's geolocation. A “query,” as used in this disclosure, is any search term used to retrieve information relating to edible 124, from a database, such as edible database 128. For instance and without limitation, computing device 104 may utilize a user's geolocation that specifies a user is located in Anchorage, Alaska to generate a query containing “Anchorage, Alaska” to identify a plurality of edibles 124 available within Anchorage, Alaska. Computing device 104 displays a plurality of edibles 124 within display interface 108 and receives a user selection containing an edible of interest. A user selection may include any user choice, picking one or more edibles 124 from within a plurality of edibles 124. A user selection may be received from user client device 116.

With continued reference to FIG. 1, computing device 104 receives nourishment information relating to an edible of interest. “Nourishment information,” as used in this disclosure, is data, including any numerical, character, and/or symbolic data, describing the nutritional content of edible 124. Nourishment information 132, may include information describing the contents and/or ingredients of an edible and/or the impact of edible 124 on a human body. Nourishment information 132 may include a caloric input, describing the total calorie count contained within edible 124. Nourishment information 132 may include a nutrient input, describing the total quantities of one or more nutrients contained within edible 124. For example, a nutrient input may describe the total number of carbohydrates, fats, proteins, minerals, additives, enzymes, vitamins, sugar, cholesterol, and the like contained within a specified serving of edible 124. For instance and without limitation, nourishment information 132 may specify that an edible containing a dinner option containing chicken parmesan with baked ziti contains 1200 calories in a serving size that equates to half of the dinner option. In yet another non-limiting example, nourishment information 132 may specify that a salad containing tuna fish and avocado in a green goddess dressing contains 400 calories in the entire salad, and contains 16 grams of fat, 20 grams of protein, 10 grams of carbohydrates, 6 grams of sugar, and 6 grams of fiber. In one or more embodiments, nourishment information 132 may include a plurality of ingredients wherein the plurality of ingredients are associated with edible of interest. For example, and without limitation, nourishment information may include ingredients such as flour, eggs, water and the like for an edible of interest such as bread. In one or more embodiments, nourishment information 132 may include a percentage or a fraction of each ingredient for the edible of interest. For example, and without limitation nourishment information 132 may contain information stating that for every 100 grams of bread, 33 grams and/or 33% contains flour. Nourishment information 132 may be obtained from third party device 136 and stored within edible database 128. Third party device 136, may include any device suitable for use as user client device 116, as described above in more detail. An entry containing nourishment information 132 may be generated by a meal maker who prepares and/or cooks edible 124, one or more experts in the field of nutrition and nourishment such as scientists, dieticians, nutritionists, researchers, clinicians, medical professionals and the like. Information relating to nourishment information 132 may be updated in real time, using any network methodology as described herein. Information pertaining to nourishment information 132 may be stored within edible database 128.

With continued reference to FIG. 1, computing device 104 may determine nourishment information 132, using a dietary classifier. A “classifier,” as used in this disclosure, is a process in which computing device 104 sorts inputs into categories or bins of data. A classifier may be generated using a classification process, including a classification algorithm. Classification may be performed using any of the classification processes and/or classification algorithms as described in U.S. Nonprovisional application Ser. No. 16/699,616 filed on Nov. 30, 2019, and entitled “METHODS AND SYSTEMS FOR INFORMING FOOD ELEMENT DECISIONS IN THE ACQUISITION OF EDIBLE MATERIALS FROM ANY SOURCE,” the entirety of which is incorporated herein by reference. A “dietary classifier,” as used in this disclosure, is a classifier that uses an edible of interest as an input and outputs, a dietary label using a classification process. A “dietary label,” as used in this disclosure, identifies one or more dietary patterns and/or ways of eating that edible 124 may fulfill. For example, a dietary label may indicate that edible 124 containing quinoa linguine cooked with olive oil, basil and tomatoes and topped with shrimp fulfills a Mediterranean diet, a gluten free diet, a dairy free diet, a pescatarian diet, and the like. Dietary classifier may be trained using training data. Training data, as used herein, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name and/or a description of a medical condition or therapy may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data may be obtained from expert inputs, previous iterations of generating a classification process, and the like.

Continuing to refer to FIG. 1, computing device may be configured to evaluate each ingredient of the plurality of ingredients within nourishment information 132. In one or more embodiments, evaluating each ingredient of the plurality of ingredients includes calculating a nutrient biodiversity score 134 for at least a nutrient. at least a processor and/or computing device 104 nay further be configured to evaluate each ingredient of the plurality of ingredients. Evaluating each ingredient of the plurality of ingredients includes extracting at least a nutrient from the ingredient. Ingredients are nutrition elements, wherein a “nutrition element,” as used in this disclosure, is an item that includes at least a nutrient intended to be used and/or consumed by a user. A “nutrient,” as used in this disclosure, is a biologically active compound substance whose consumption provides nourishment essential for growth and the maintenance of life in a microbiome. Types of at least a nutrient may include, without limitation, carbohydrates, proteins, fats, vitamins, minerals, dietary fiber, water, or the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron. A “nutrient biodiversity score” is data including any character, symbolic, and/or numerical data, reflecting the diversity of the nutrient compared to the user's current diet and health status; in other words, the score relays how diverse the nutrient is amongst the nutrients the user would normally eat in their diet. Nutrient biodiversity score 134 represents the effect of the at least a nutrient on the diversity of a microbiome. Nutrient biodiversity score 134 may be transient and/or dynamic. Nutrient biodiversity score 134 may be updated based on one or more meals that a user consumed and/or is planning to consume. Nutrient biodiversity score 134 may be calculated by at least a processor or computing device 104 by retrieving information contained within edible of interest and/or nourishment information. Nutrient biodiversity score 134 may be graded on a continuum, where a score of zero may indicate a user who is in extremely poor nutritional health while a score of 100 may indicate a user who is in excellent nutritional health. Nutrient biodiversity score 134 may be calculated from one or more factors that may be stored within a database such as food intake, water intake, supplement intake, prescription medication intake, fitness practice, health goals, chronic health conditions, acute health conditions, spiritual wellness, meditation practice, stress levels, and the like.

With continued reference to FIG. 1, calculating a nutrient biodiversity score 134 may include the use of a biodiversity machine-learning model. Calculating nutrient biodiversity score 134 also includes retrieving at least a nutrient containing a logged biodiversity entry. At least a processor may then generate a biodiversity machine-learning model, wherein the biodiversity machine-learning model utilizes the logged nourishment entry as an input, and outputs the nutrient biodiversity. A “logged nourishment entry,” as used in this disclosure, is any stored factor that is utilized to calculate a nutrient biodiversity score 134. A logged nourishment entry may include a user's daily water intake, a user's supplement intake, and the like as described below in more detail. A logged nourishment entry may include a nourishment behavioral target. A “nourishment behavioral target,” as used in this disclosure, is a user behavior goal relating to nourishment possibilities. A behavior goal may include a desire to cook a certain number of meals at home each week, or a desire to only eat fast food a certain number of times each month. A behavior goal may be self-reported by a user, and a user's progress towards meeting the behavior goal may be calculated into a user's nutrient biodiversity score 134. For example, a user who continues to not achieve any progress towards a user's nourishment behavior target to eat fish at least three times each week may decrease a user's overall nutrient biodiversity score 134, while a user with the same nourishment behavior target and who does continuously eat fish three times each week may increase the user's overall nutrient biodiversity score 134. A behavior goal may relate to a food source, such as a desire to only go out to eat no more than three days each week and to eat the rest of a user's meals at home. A behavior goal may relate to a food option such as to only consume foods that do not contain genetically modified organisms, or to only consume foods that do not contain high fructose corn syrup. Nutrient biodiversity score 134 may be affected by, without limitation, seasonal nutrients, allergies, local nutrients, or the like. In one or more embodiments, one or more logged nourishment entries may be stored in a database as described herein. At least a processor may retrieve one or more elements of data containing a logged nourishment entry such as by generating a query, including any of the queries as described herein. At least a processor and/or computing device 104 generates a biodiversity machine-learning process. A “machine-learning process,” as used in this disclosure, is a process that automatedly uses a body of data known as “training data” and/or a “training set” to generate an algorithm that will be performed by at least a processor and/or a module to produce outputs given data provided as inputs; this is in contrast to a non-machine learning software program where the commands to be executed are determined in advance by a user and written in a programming language. As used herein, training data used to train biodiversity machine-learning model is biodiversity training data. Biodiversity training data may input any of the data described herein from proposed user selection relating to nourishment or a database, and outputs a biodiversity nutrient score. A “biodiversity machine-learning process,” as used in this disclosure, is any machine-learning process that utilizes a logged nourishment entry as an input, and outputs a biodiversity nutrient score 134. “Training data,” as used in this disclosure, is data containing correlations that a machine-learning process including a machine-learning algorithm and/or machine-learning process may use to model relationships between two or more categories of data elements. Training data may be formatted to include labels, for instance by associating data elements with one or more descriptors corresponding to categories of data elements. Training data may not contain labels, where training data may not be formatted to include labels. Biodiversity machine-learning process may be generated calculating one or more machine-learning algorithms and/or producing one or more machine-learning models.

With continued reference to FIG. 1, a machine-learning model may include one or more supervised machine-learning algorithms, which may include active learning, classification, regression, analytical learning, artificial neural network, backpropagation, boosting, Bayesian statistics, case-based learning, genetic programming, Kernel estimators, naïve Bayes classifiers, maximum entropy classifier, conditional random field, K-nearest neighbor algorithm, support vector machine, random forest, ordinal classification, data pre-processing, statistical relational learning, and the like. A machine-learning algorithm may include an unsupervised machine-learning algorithm, that is trained using training data that does not contain data labels. An unsupervised machine-learning algorithm may include a clustering algorithm such as hierarchical clustering, k-means clustering, mixture models, density based spatial clustering of algorithms with noise (DBSCAN), ordering points to identify the clustering structure (OPTICS), anomaly detection such as local outlier factor, neural networks such as autoencoders, deep belief nets, Hebbian learning, generative adversarial networks, self-organizing map, and the like. A machine-learning algorithm may include semi-supervised learning that may be trained using training data that contains a mixture of labeled and unlabeled data. A machine-learning algorithm may include reinforcement learning, self-learning, feature learning, sparse dictionary learning, anomaly detection, robot learning, association rules, and the like. A machine-learning algorithm may include generating one or more machine-learning models.

Still referring to FIG. 1, calculating a nutrient biodiversity score may further include the use of a biodiversity classifier. Biodiversity classifier may be configured to output at least a datum that labels or otherwise identifies a set of data that are clustered together, found to be close under a distance metric as described below, or the like. At least a processor and/or another device may generate a biodiversity classifier using a classification algorithm, defined as a processes whereby a at least a processor derives a classifier from training data. Biodiversity classifier may receive at least a nutrient as input and output a nutrient biodiversity score 134. Training data for biodiversity classifier may include any of the inputs and outputs described above. Training data for biodiversity classifier may include nutrients correlated to nutrient biodiversity scores. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, at least a processor may be configured to generate a biodiversity classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)-P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. At least a processor may then calculate a likelihood table by calculating probabilities of different data entries and classification labels. At least a processor may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

Still referring to FIG. 1, at least a processor is also configured to optimize the plurality of ingredients within edible of interest and/or nourishment information 132 as a function of each nutrient biodiversity score 134. As used herein, “optimize” means to rearrange or rewrite to improve efficiency of processing. Optimizing plurality of ingredients may include creating a list of the plurality of ingredients and organizing list of the plurality of ingredients based on the nutrient biodiversity score 134 of each ingredient. Organizing a list of plurality of ingredients may include arranging them in order from lowest nutrient biodiversity score 134 to highest nutrient biodiversity score 134, or vice versa. Additionally, without limitation, organizing the list may include organizing the list from most present in the user's microbiome to least present or vice versa. List of ingredients may be organized in anyway in order to figure out which ingredients will increase the biodiversity of the user's microbiome.

Referring still to FIG. 1, at least a processor is further configured to adjust the plurality of ingredients as a function of the optimization of the plurality of ingredients. Ingredient adjustment is an action of adjusting the plurality of ingredients. As used herein, an “adjustment” is any change in the original plurality of ingredients, including, without limitation, a change in amounts of ingredients, exchange of ingredients, or the like. Adjusting plurality of ingredients may include replacing at least an ingredient of the plurality of ingredients. Also, adjusting plurality of ingredients may further include maximizing the diversity of nutrients in the plurality of ingredients.

Furthermore and still referring to FIG. 1, at least a processor may use a language processing module at any of the steps explained herein. A language processing module may include any hardware and/or software module. A language processing module may be configured to extract, from the one or more documents, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols. including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model

A language processing module may operate to produce a language processing model. Language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring to FIG. 1, a language processing module and/or diagnostic engine may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors. Nutrient biodiversity score may be described in further detail in Nonprovisional application Ser. No. 17/833,365 having attorney docket number 1057-205USU1 filed on Jun. 6, 2022 and entitled “AN APPARATUS AND METHOD FOR ADJUSTING A USER NOURISHMENT SELECTION BASED ON NUTRIENT DIVERSITY,” the entirety of which is incorporated herein by reference.

With continued reference to FIG. 1, computing device may be configured to determine a nutritional requirement 138 as a function of at least the nourishment information. A “nutritional requirement,” as used in this disclosure, is data, including any character, symbolic, and/or numerical data, reflecting the current overall nutritional impact of a meal, snack, and/or drink for a specific group of human profiles and/or representative profiles. A nutritional requirement 138 may be transient and/or dynamic and varies based on ingredients utilized, recipe, cooking instructions, storage impacts, meal size, drink size, snack size, and the like. A nutritional requirement 138 may be graded on a continuum, where a score of zero may indicate a meal, snack, and/or drink which has an extremely poor nutritional impact for a human profile, while a score of 100 may indicate a meal, snack, and/or drink which has an excellent nutritional impact for a human profile. A negative nutritional requirement 138 may reflect a meal, snack, and/or drink that has no beneficial nutritional impact for a human profile and may have a net detrimental impact. In such an instance, a negative nutritional requirement may not contain a numerical assignment. In one or more embodiments, nutritional requirement 138 may include a scoring of a food item wherein the scoring denotes the health impact of the food. In one or more embodiments, food items with higher scoring may be associated with a greater positive impact in comparison to food items with lower scoring.

With continued reference to FIG. 1, computing device 104 may be configured to calculate and/or determine using a machine learning process a nutritional requirement 138 of a meal option or edible of interest using a training set, wherein the machine-learning process uses a meal option, edible of interest and/or associated nourishment information as an input, and outputs a nutritional requirement 138. A “machine learning process,” as used in this disclosure, is a process that automatically uses a body of data known as “training data” and/or a “training set” to generate an algorithm that will be performed by computing device 104 and/or any module to produce outputs given data provided as inputs; this is in contrast to a non-machine learning software program where the commands to be executed are determined in advance by a user and written in a programming language. A machine-learning process may be implemented, without limitation, as described in U.S. Nonprovisional application Ser. No. 16/502,835, filed on Jul. 3, 2019, and entitled “METHODS AND SYSTEMS FOR ACHIEVING VIBRANT CONSTITUTION BASED ON USER INPUTS,” the entirety of which is incorporated herein by reference. A machine-learning process may include generating one or more machine-learning models. A machine learning model includes any mathematical representation of a machine-learning process. A machine learning model may include generating one or more machine-learning algorithms. A machine-learning algorithm may include supervised machine-learning algorithms, unsupervised machine-learning algorithms, lazy learning algorithms, and the like. A machine-learning algorithm utilizes training set to identify patterns in training set so that inputs of a machine-learning algorithm correspond to target outputs of the machine-learning process. A machine-learning algorithm may include one or more machine-learning algorithms, including but not limited to, regression, classification, target, feature, label, overfitting, regularization, parameter and hyper-parameter, and the like.

With continued reference to FIG. 1, computing device 104 may determine a nutritional requirement 138 of a meal option, edible of interest and/or associated nourishment information 132 as a function of machine-learning process. Computing device 104 displays a nutritional requirement 138 within display interface 108. Nutritional requirement 138 includes a range of values. For example, a meal option, edible of interest and/or associated nourishment information 132 containing shrimp scampi served on a bed of linguini may contain a nutritional requirement 138 that includes a range of values ranging between 52-75. In such an instance, a range of values may be compared to the standard range, which may be from between 0 to 100, where 0 indicates a meal option that has an unsatisfactory nutritional impact, while a score of 100 indicates a meal option that has a satisfactory nutritional impact. A range of values may aid a user in making informed decisions about a meal option, edible of interest and/or associated nourishment information 132, as well as to compare a first meal option, edible of interest and/or associated nourishment information 132 to a second meal option, edible of interest and/or associated nourishment information 132. For example, a nutritional requirement 138 for a first meal option, edible of interest and/or associated nourishment information 132 containing filet mignon with sauteed spinach may contain a first nutritional requirement 138 that ranges between 77-92, while a nutritional requirement 138 for a second meal option, edible of interest and/or associated nourishment information 132 containing fried chicken with French fries may contain a second nutritional requirement 138 that ranges between 7-12. In such an instance, a user may compare ranges between a first nutritional requirement 138 and the ranges of a second nutritional requirement 138, to make an informed decision that the first meal option, edible of interest and/or associated nourishment information 132 will have an overall more positive impact on a user's health as compared to the second meal option, edible of interest and/or associated nourishment information 132.

With continued reference to FIG. 1, in one or more embodiments, computing device 104 may receive a user input identifying a food preference. A “food preference,” as used in this disclosure, is a label identifying a dietary choice and/or pattern of eating. A food preference may identify particular foods that a user likes and/or dislikes. For example, a food preference may identify that a user likes to consume turkey breast, avocado, mesclun greens, and shrimp, while the user dislikes to consume kale, cauliflower, and Brussel sprouts. A food preference may identify a particular pattern of eating that a user follows, for example, a user who follows a paleo diet or a vegetarian diet. A food preference may identify one or more foods that a user is unable to consume due to an allergy, intolerance, ethical reasons, and/or any other reason that prohibits the user from consuming the food. For example, a food preference may specify that a user does not consume any wheat containing products because the user is intolerant to wheat. Computing device 104 displays a nutritional requirement 138, as a function of a food preference. For example, a food preference that specifies a user follows a ketogenic diet, may be utilized to display a nutritional requirement 138 of a meal option, edible of interest and/or associated nourishment information 132 for a user following the ketogenic diet. In yet another non-limiting example, a food preference that specifies a user has an allergy to avocado may be utilized to display a nutritional requirement 138 of a meal option, edible of interest and/or associated nourishment information 132 for a user with an allergy to avocado. In one or more embodiments, user dietary habit may include a food preference. Nutritional requirement 138 may be described in further detail in Nonprovisional application Ser. No. 16/919,532 having attorney docket number 1057-109USU1 filed on Jul. 20, 2020 and entitled “METHODS AND SYSTEMS FOR CALCULATING NUTRITIONAL REQUIREMENTS IN A DISPLAY INTERFACE” the entirety of which is incorporated herein by reference.

With continued reference to FIG. 1, computing device 104 is configured to generate a score machine-learning process. A “machine-learning process,” as used in this disclosure, is a process that automatedly uses training data to generate a model and/or an algorithm, that will be performed by computing device 104, to produce outputs given data provided as inputs; this is in contrast to a non-machine learning software program where the commands to be executed are determined in advance by a user and written in a programming language. An “score machine-learning process,” as used in this disclosure, uses a performance profile 112 and nourishment information 132 relating to an edible of interest as an input, and outputs an edible score. Generating a score machine-learning process 132 includes training score machine-learning process 132 using edible training data 144. “Edible training data,” as used in this disclosure, is training data containing a plurality of data entries containing elements of a performance profile and nourishment information relating to edible 124, correlated to an edible score. Edible training data 144 may be obtained from expert inputs and/or previous iterations of generating score machine-learning process 140. Score machine-learning process 140 may be implemented as any machine learning process as described in U.S. Nonprovisional application Ser. No. 16/372,512 filed on Apr. 2, 2019 and entitled “METHODS AND SYSTEMS FOR UTILIZING DIAGNOSTICS FOR INFORMED VIBRANT CONSTITUTIONAL GUIDANCE,” the entirety of which is incorporated herein by reference.

With continued reference to FIG. 1, score machine-learning process 140 outputs an edible score 148. An “edible score,” as used in this disclosure, is data, including any character, symbolic, and/or numerical data, containing a score reflecting the nutritional impact of an edible on a user's body and/or health. An edible score 148 may be transient and/or dynamic. An edible score 148 may be graded on a continuum, where a score of zero may indicate an edible that will have an extremely poor nutritional impact on a user, while a score of 100 may indicate an edible that will have an excellent nutritional impact on a user. An edible score may be updated based on a serving size of an edible.

With continued reference to FIG. 1, computing device 104 may be configured to calculate a first edible score 148 for a first edible 124, calculate a second edible score 148 for a second edible 124, and chart the first edible score 148 as a function of the second edible score 148. Charting may include mapping and/or graphing a first edible score 148 as compared to a second edible score 148. For instance and without limitation, a first edible 124 that contains a first edible score 148 of 74 for a first edible 124 containing grilled salmon served with rice pilaf and steamed broccoli may be charted on a graph versus a second edible 124 that contains a second edible score 148 of 22 for a second edible 124 containing fried chicken served with mashed potatoes and gravy. A chart may be displayed within display interface 108 for a user to view a first edible score charted as a function of a second edible score. In an embodiment, edible score 148 may expressed using negative values, such as −100 to −1 to illustrate a detrimental impact an edible may have on a user, wherein −100 may be the most extreme detrimental impact and −1 may the lest extreme detrimental impact. A score of 0 may refer to a neutral impact, while 1-100 may refer to the level of positive impact an edible has on a user as described further below. On a scale of −100 to 100, scores between −100 to −1 may be referred to as a detriment range or detrimental score, and scores between 1-100 may referred to as a fueling range or fueling score. In some embodiments, a score of −10 to 10 may represent a neutral impact. In some embodiments, a score of −20 to 20 may represent a neutral impact. In some embodiments a score of 20 and above may represent a positive impact. In some embodiments a score of 40 and above may represent a positive impact. In some embodiments a score of 50 and above may represent a positive impact. In some embodiments a score of −20 and below may represent a negative impact. In some embodiments a score of −40 and below may represent a negative impact. In some embodiments a score of −50 and below may represent a negative impact. In some embodiments, a score representing a positive impact may be referred to as a “fueling score.” A fueling score means that the edible will fuel the body and/or provide nutrients.

Still referring to FIG. 1, in some embodiments computing device 104 may be configured to identify trends in consumption of edibles correlated to the timestamps in order to determine an edible score 148. Trends may reflect the impact edible 124 has on a user based on the time of day and time of consumption. Trends may be positive, neutral, and negative. A positive trend may refer to a beneficial impact edible 124 has on a user on a user's health. A positive trend may refer to an optimal impact edible 124 has on user's health. A negative trend may refer to a detrimental impact edible 124 has on a user on a user's health. A neutral trend may refer to an adequate or no impact edible 124 has on a user on a user's health. For example, computing device 104 may derive from nourishment information 132 and/or edible database 128, that tart cherry juice has a high level of melatonin. Computing device 104 may identify that consumption of the juice at 8 am in the morning has a negative impact on user as it may take energy away, slow metabolism down, and the like. Computing device 104 may identify that consumption of the juice at 5 pm has a neutral impact on a user, while consumption of the juice 8 pm has a positive and optimal impact on the user's health as a sleeping aid. Trends in consumption may be identified using the logged performance metrics and biological extraction retrieved from performance profile 112, and any other data as described throughout this disclosure. For example, physiological state data may be received after consumption of the tart cherry juice as logged by the user. Computing device 104 may generate a trend classifier configured to receive the performance profile 112 and nourishment information 132 as inputs and output a plurality of trends associated with an edible. Trend classifier may be trained by a trend training data set correlating timestamps and nourishment information 132 to a plurality of trends. In some embodiments, the output of the trend classifier may be used in the edible training data set as described above.

Still referring to FIG. 1, in some embodiments, edible score 148 may additionally be based on a timestamp of consumption, trends in consumption, outputs of the trend classifier, and the like. The nutritional impact of edible score 148 may be based on a time of consumption correlated to an identified trend as described above. Edible score 148 may fluctuate and/or be updated based on the time of day the edible of interest is received. In some embodiments, edible score 148 may be used as an indication of time windows an edible 124 should be consumed to gain a positive or optimal result. For example, computing device may output a plurality of edible scores 148 for an edible of interest to be displayed through display interface 108, wherein each edible score is associated/displayed with a correlating timestamp. For example, on a scale of 1-100 as described above, tart cherry juice may be displayed as scoring at 10 at 8 am, 50 at 5 pm and 100 at 8 pm. In some embodiments, computing device 104 may display text recommending optimal time ranges for consumption of an edible. For example, computing device 104 may generate text stating tart cherry is recommended to be consumed from 5 pm to 12 am, or should be avoided from 8 am to 1 pm.

Still referring to FIG. 1, computing device 104 may be configured to display compatible edibles based on the edible score 148. A “compatible edible,” as used herein, is a substitute edible. When edible score 148 is average or low, representing an edible of interest to have a negative or neutral impact on user's health, computing device 104 may identify a plurality of compatible elements that have a positive impact on a user to substitute the edible of interest with. Substitution may incorporate nourishment information 132 of the edible of interest. For example, computing device 104 may retrieve from edible database 128 edibles that are similar in calorie count, flavor, vitamins, texture, and the like. Computing device 104 may identify a compatible element using a compatible classifier configured to receive edible 124 and output edible 124 classified to a plurality of nutrient information based categories. Nutrient information based categories may relate to calories, flavor, texture, vitamins, minerals, protein, fats, and the like. For example, categories may include “high protein,” “low sodium,” “spicy,” and the like. Compatible classifier may be trained using a compatible training data set correlating an edible of interest to a plurality of nutrient information based categories. Computing device 104 may take the classified edible from the compatible classifier and select a compatible edible from edible database 128 that belongs to the same or similar nutrient information based categories.

Still referring to FIG. 1, edible score 148 may be based on precision nourishment. “Precision nourishment,” as used herein is, is a state of optimal consumption of nutrients. Precision nourishment may be based on accuracy of the nutritional impact on a user's physiological state. Achieving precision nourishment may include narrowing the ranges for edible score as to what is considered positive, optimal, negative, and neutral in nutritional value and time of consumption by a user based on their physiological state data. As a non-limiting example, edible score range for what is considered positive may be reduced from 1-100 to 20-100, 40-100, 50-100, and the like. These ranges may be tailored, adjusted, or narrowed, based on trends identified in a user's physiological state over of period of time using system 100. For example, computing device 104 may receive feedback of edible 124 consumed by a user that was scored or recommended by system 100. Feedback may be in the form of updates to biological extractions and psychological state data, such as, digestive system, circulatory system, levels of age-related degradation, and the like. Feedback may be in the form of updates to information in performance profile 122 and nourishment information 132. Computing device 104 may assess signs of positive, neutral, or negative trends in the feedback using a machine-learning process. For example, computing device 104 may train a precision classifier configured to receive feedback as an input and output a plurality of trends. A precision training data set may correlate the feedback to a plurality of trends. For example, computing device 104 may receive a user's biological extraction or physiological state data, nourishment information 132, and performance profile 112 after consumption of nutrients over a month by a user and match the nourishment information, time of consumption, and physiological state data to a positive trend, wherein the positive trend may be progress in reaching a dietary goal or health progress in a user physiological state. Precision classifier training data may correlate elements of performance profile, nourishment information, edibles score, and the like to a positive negative, optimal or neutral trend. For example, high levels of vitamin D in a user with an iron deficiency may be correlated to a positive trend wherein computing device 104 matches the time of consumption of iron enriched foods consumed by the user. Computing device may take the trend outputs and continuously use the trends as training data for machine-learning processes as described above to fine tune the generation of edible score 108 and edible recommendations.

With continued reference to FIG. 1, computing device 104 may receive a logged performance metric from a plurality of logged performance metrics associated with the edible of interest and display the logged metric through a display interface. In one or more embodiments, a database may include a plurality of logged performance metrics as described above wherein each performance metric may include information indicating an individual's reaction to a particular edible of interest. In one or more embodiments, each logged performance metric may include information indicating an individual's physiological response to an edible of interest. In one or more embodiments, computing device 104 may select a logged performance metric associated with the determined edible of interest as described above and display the logged performance metric through display interface 108. In one or more embodiments, similar individuals with similar performance profiles as the current user may have consumed edibles in the past and have had various physiological effects. In one or more embodiments, computing device may find an individual having a similar performance profile and receive the logged performance metric associated with the consumed edible or food item. In one or more embodiments, the logged performance metric may include the edible consumed and the various physiological and/or emotional responses associated with the consumption of the edible item. In one or more embodiments, a database may be populated with a plurality of logged performance metric associated with a plurality of performance profiles wherein each logged performance metric may be associated with a particular edible of interest and/or food item that has been previously ingested and recorded. In one or more embodiments, computing device may search database for one or more logged performance metrics associated with edible of interest and display the one or logged performance metrics to a user through display interface. In one or more embodiments, display of logged performance metric may allow a user to view the various physiological effects of edible of interest on other individuals. In one or more embodiments, computing device 104 may receive one or more logged performance metrics and select a logged performance metric associated with a performance profile similar to performance profile 112 of user. In one or more embodiments, similarities may include similar diseases, similar physiological attributes between two performance profiles and the like. In one or more embodiments, computing device may select one or more logged performance metrics associated with the edible of interest and select a logged performance metric associated with an individual who has a similar biological extraction, performance profile and the like as the current user. In one or more embodiments, computing device 104 may select the performance profile having the highest degree of match wherein the highest degree of match may indicate a performance profile having the most similarities with performance profile 112 in comparison to other profiles. In one or more embodiments, computing device 104 may select a performance profile based on particular elements within performance profile 112 such as but not limited to, age, gender, weight, disease state and the like. In one or more embodiments, the logged performance metric may be displayed through display interface 108 such that a user may be informed on how an edible of interest has affected other similar individuals.

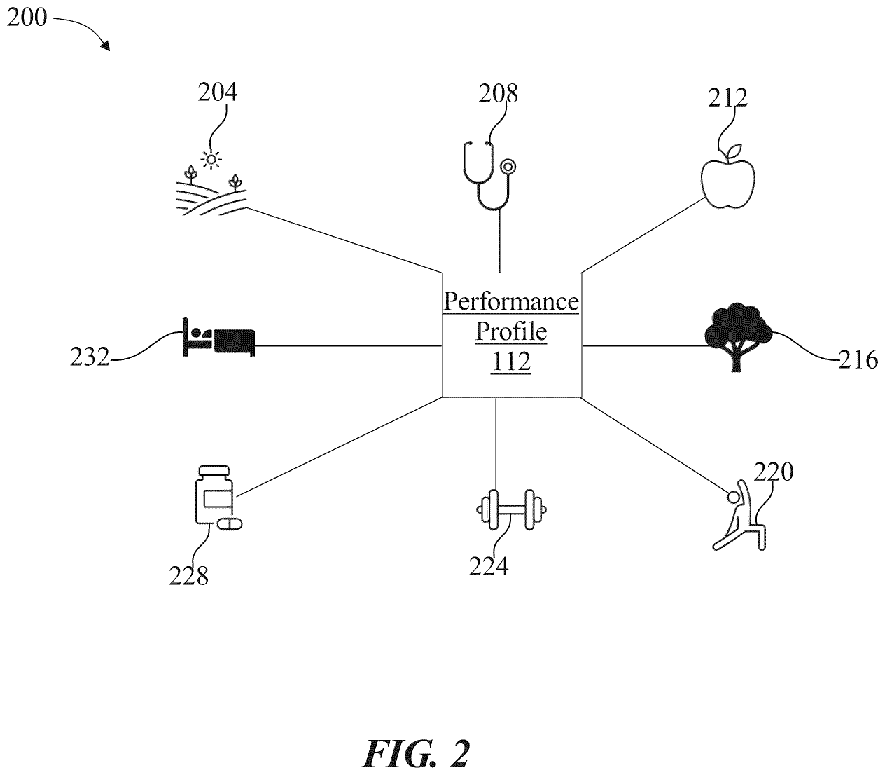

Referring now to FIG. 2, an exemplary embodiment 200 of performance profile 112 is illustrated. Performance profile 112 may include one or more elements of data relating to a user, as described above in more detail in reference to FIG. 1. A performance profile 112, may include an element of data 204 relating to a user's access to nature and fresh air and/or how many hours a day a user spending indoors under artificial lights. For example, a performance profile 112 may indicate that a user spends on average thirty minutes outdoors every morning before heading to work. A performance profile 112, may include an element of data 208 relating to a user's access to medical care and medical services. For example, a performance profile 112 may contain a biological extraction from a user, such as findings from a CAT (CT) scan, showing a complex bone fracture. A performance profile 112, may include an element of data 212 relating to a user's current eating habits. For example, a performance profile 112 may contain an indication that a user dislikes all pork containing products, and the user likes all poultry containing products. A performance profile 112 may include an element of data 216 relating to a user's toxicity levels, including any of the toxicity levels as described above in more detail in reference to FIG. 1. For example, a performance profile 112 may contain an indication that a user has high urinary levels of cadmium. A performance profile 112, may include an element of data 220 relating to a user's stress levels, meditation practice, and/or overall body flexibility. For example, a performance profile 112 may contain an indication that a user practices hatha yoga three days each week. A performance profile 112 may include an element of data 224 relating to a user's fitness and/or activity level. For example, a performance profile 112 may contain an indication that a user engages in three days of biking each week, and two days of strength training each week. A performance profile 112 may include an element of data 228 relating to a user's medication and/or supplement use. For example, a performance profile 112 may contain an indication that a user takes a prescription medication for osteoporosis, and an iron containing supplement. A performance profile 112 may include an element of data 232 relating to a user's sleeping patterns and sleeping habits. For example, a performance profile 112 may contain an indication that over the past week the user has slept an average of six and a half hours each night and taken an average of twenty-two minutes to fall asleep each night.

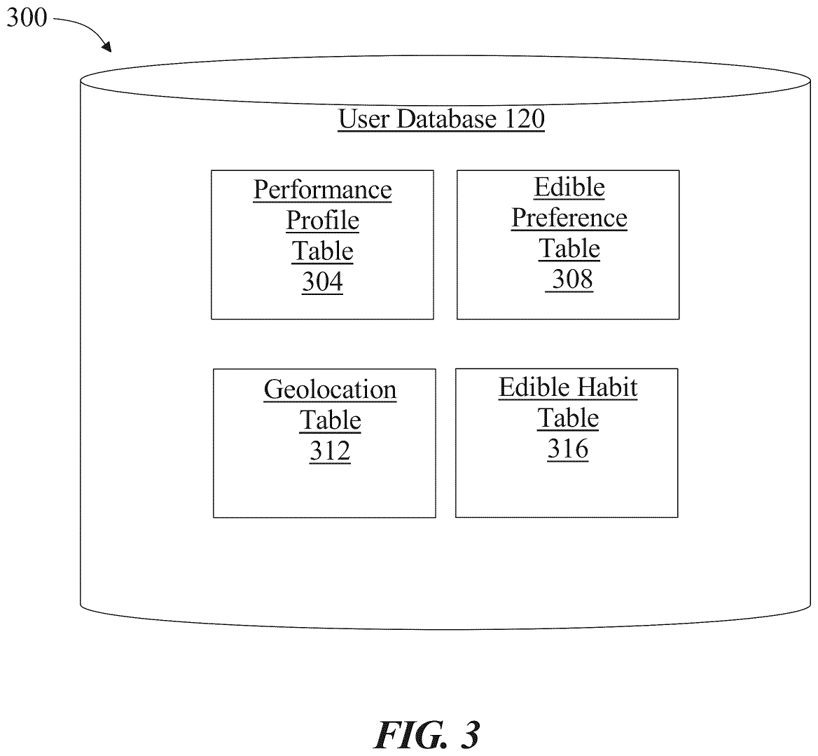

Referring now to FIG. 3, an exemplary embodiment 300 of user database 120 is illustrated. User database 120 may be implemented as any data structure suitable for use as described above in more detail in reference to FIG. 1. One or more tables contained within user database 120 may include performance profile table 304; performance profile table 304 may include any information relating to a user's performance profile 112. For instance and without limitation, performance profile table 304 may include information describing a user's sleeping patterns over the prior week, including information specifying how much sleep a user got each night, as well as how many rapid eye movement (REM) cycles a user entered. One or more tables contained within user database 120 may include edible preference table 308; edible preference table 308 may include information describing a user's food preferences. For instance and without limitation, edible preference table 308 may indicate that a user likes foods that are spicy, but the user does not like meals that contain cucumbers. One or more tables contained within user database 120 may include geolocation table 312; geolocation table 312 may include any indication as to a user's geolocation. For instance and without limitation, geolocation table 312 may specify that a user is currently located in Bangor, Maine. One or more tables contained within user database 120 may include edible habit table 316; edible habit table 316 may include information describing a user's edible habits. For instance and without limitation, edible habit table 316 may specify that a user skips breakfast every morning, but eats lunch and dinner, and one afternoon snack.

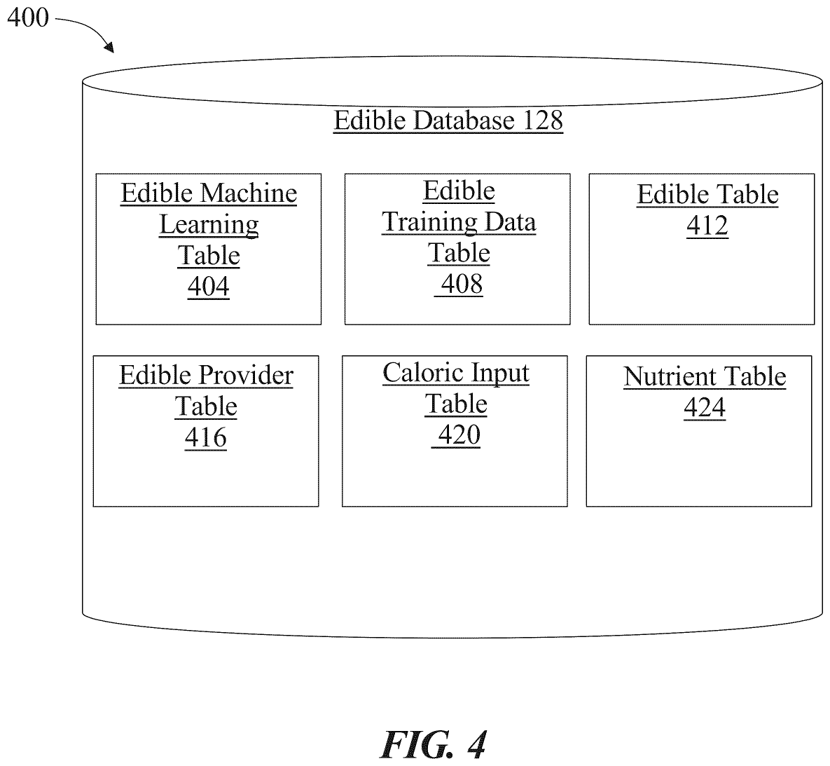

Referring now to FIG. 4, an exemplary embodiment 400 of edible database 128 is illustrated. Edible database 128 may be implemented as any data structure as described above in more detail in reference to FIG. 1. One or more tables contained within edible database 128 may include score machine-learning table 404; score machine-learning table 404 may include information relating to score machine-learning process 140. One or more tables contained within edible database 128 may include edible training data table 408; edible training data table 408 may include information relating to one or more edible training data sets 144. One or more tables contained within edible database 128 may include edible table 412; edible table 412 may include information relating to one or more edibles 124. One or more tables contained within edible database 128 may include edible provider table 416; edible provider table 416 may include information relating to one or more edible providers, such as a meal maker. One or more tables contained within edible database 128 may include caloric input table 420; caloric input table 420 may include information relating to the caloric input of one or more edibles 124. One or more tables contained within edible database 128 may include nutrient table 424; nutrient table 424 may include information relating to the nutrient input of one or more edibles 124.

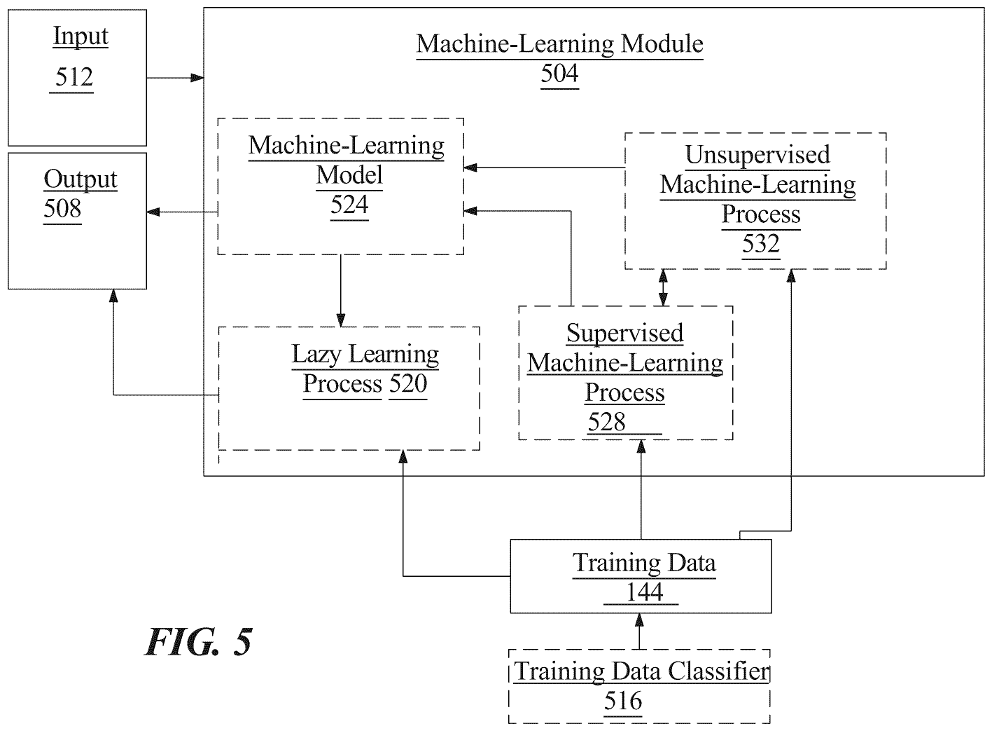

Referring now to FIG. 5, an exemplary embodiment 500 of a machine-learning module 504 that may perform one or more machine-learning processes as described in this disclosure, is illustrated. Machine-learning module may perform determinations, classification, and/or analysis steps, methods, processes, or the like as described in this disclosure using machine learning processes, including for example, score machine-learning process 140. A machine learning process includes any of the machine-learning processes as described above in more detail in reference to FIG. 1. Machine learning module 504 uses edible training data 144 to generate an algorithm that will be performed by computing device 104 and/or machine-learning module 504 to produce outputs 508 such as edible score 148 given data provided as inputs 512, such as performance profile 112 and/or edible 124.

With continued reference to FIG. 5, edible training data 144 may include any of the edible training data 144 as described above in more detail in reference to FIG. 1. Multiple data entries in training data 144 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 144 according to various correlations; correlations may indicate causative and/or predictive links between categories of data elements, which may be modeled as relationships such as mathematical relationships by machine-learning processes as described in further detail below. Training data 144 may be formatted and/or organized by categories of data elements, for instance by associating data elements with one or more descriptors corresponding to categories of data elements. As a non-limiting example, training data 144 may include data entered in standardized forms by persons or processes, such that entry of a given data element in a given field in a form may be mapped to one or more descriptors of categories. Elements in training data 144 may be linked to descriptors of categories by tags, tokens, or other data elements; for instance, and without limitation, training data 144 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 144 may include one or more elements that are not categorized; that is, training data 144 may not be formatted or contain descriptors for some elements of data. Machine-learning algorithms and/or other processes may sort training data 144 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 144 to be made applicable for two or more distinct machine-learning algorithms as described in further detail below. Training data 144 used by machine-learning module 504 may correlate any input data as described in this disclosure to any output data as described in this disclosure. As a non-limiting illustrative example machine-learning module 504 may utilize performance profile 112 and/or edible 124 as an input and output an edible score 148.

Further referring to FIG. 5, edible training data 144 may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 504 may generate a classifier using a classification algorithm, defined as a processes whereby a computing device and/or any module and/or component operating thereon derives a classifier from edible training data 144. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to match certain elements of data contained within performance profile 112. For example, training data classifier 516 may characterize a sub-population such as a cohort of persons that contain user inputs that include sleeping patterns, or user inputs that contain toxicity levels. Training data classifier 516 may analyze items and/or phenomena contained within a performance profile 112 may be selected.

Still referring to FIG. 5, machine-learning module 504 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 144. Heuristic may include selecting some number of highest-ranking associations and/or training data 144 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate a machine-learning model. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 144 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include performance profile 112 and/or edible 124 as described above as inputs, edible score 148 as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in edible training data 144. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 504 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 5, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from an edible training data 144 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using edible training data 144.

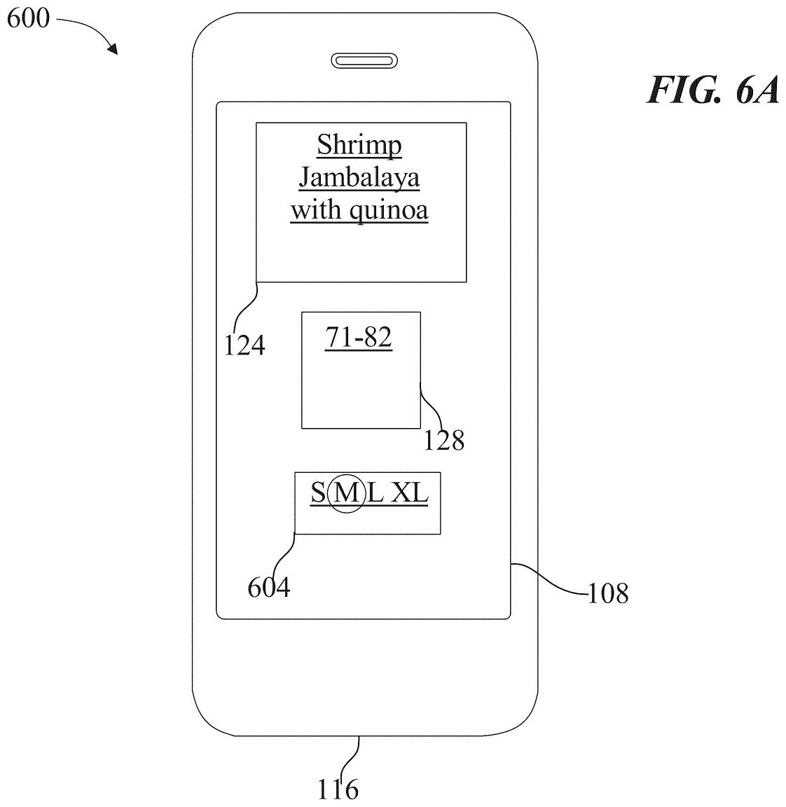

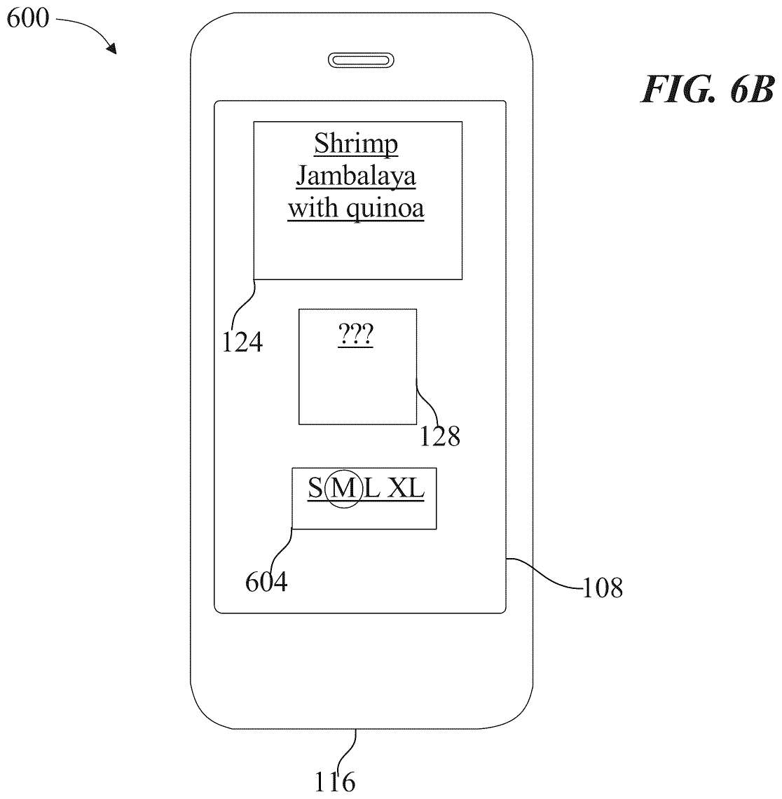

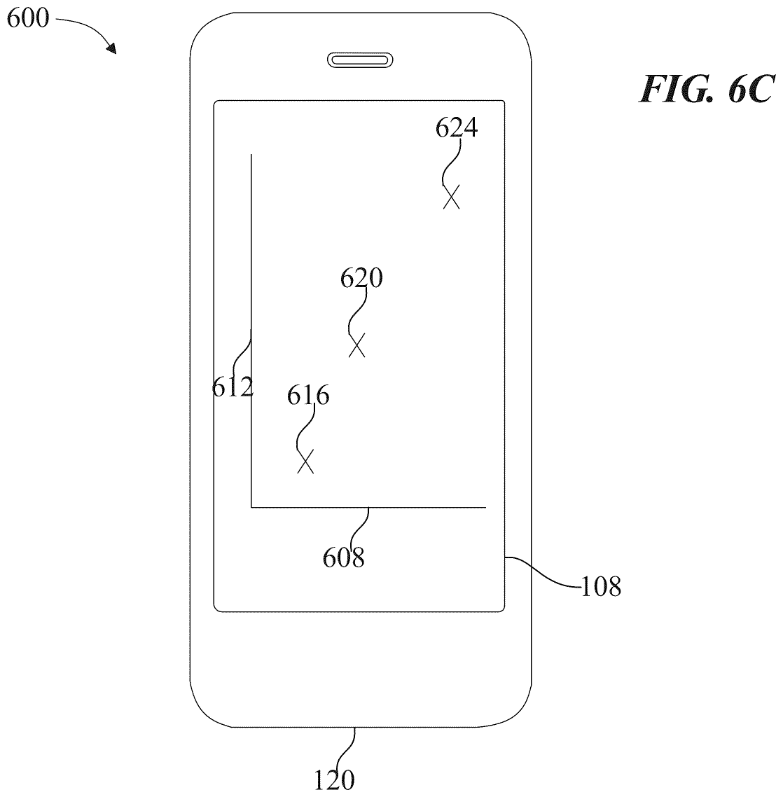

Referring now to FIGS. 6A-6C, exemplary embodiments 600 of display interface 108 are illustrated. Referring to FIG. 6A, in an embodiment, display interface 108 may be viewed by a user, on user client device 116. In an embodiment, display interface 108 may display a description of edible 124. Display interface 108 may display edible score 148 as a numerical output. In an embodiment, edible score 148 may be displayed based on a selected serving size 604 of edible 124. For example, a medium sized serving of shrimp jambalaya with quinoa may have an edible score 148 that ranges from 71-82, indicating that the edible 124, has a good nutritional impact on a user. Referring now to FIG. 6B, in an embodiment, edible score 148 may be displayed as “???” such as when edible score 148 may be unable to be calculated, such as when performance profile 112 does not contain enough information to calculate an edible score 148, or when edible 124 may no longer be available or is out of stock. Referring now to FIG. 6C, display interface 108 may display a plurality of edibles charted as a function of an edible score for each of the plurality of edibles. A chart may include a graph, depicted in a diagrammatic form, containing an “X” axis 608, and a “Y” axis 612. In an embodiment, X axis 608 may contain each of a plurality of edibles 124, charted against a Y axis 612 containing each of a plurality of edible scores 148. In such an instance, display interface 108 may display a first edible 616 containing a first edible score, a second edible 620 containing a second edible score, and a third edible 624 containing a third edible score. In such an instance, third edible 624 may be displayed at almost the top of the Y axis 612, indicating it contains a very high edible score, as compared to first edible 616, which is displayed closer to the bottom of the Y axis 612, indicating it contains a much lower edible, while second edible 620 is located around the middle of Y axis 612, indicating it has a mediocre edible score. In such an instance, display interface 108 containing a chart of various edibles and corresponding edible scores may be utilized by a user to make an informed decision as to how each edible will impact a user's body.

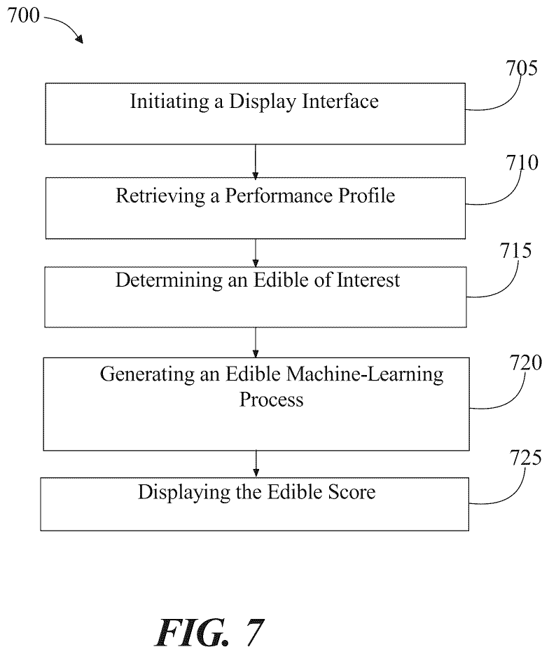

Referring now to FIG. 7, an exemplary embodiment 700 of a method of calculating an edible score in a display interface is illustrated. At step 705, computing device 104 initiates a display interface 108. Display interface 108 includes any of the display interfaces as described above in more detail in reference to FIG. 1. Display interface 108 may include a form or other graphical element having display fields, as described above in more detail.

With continued reference to FIG. 7, at step 710, computing device 104 retrieves a performance profile 112 relating to a user. A performance profile 112, includes any of the performance profiles 112 as described above in more detail in reference to FIG. 1. A performance profile 112 may include one or more elements of data relating to a user, as described above in more detail in reference to FIGS. 1-6. For instance and without limitation, a performance profile 112 may include a biological extraction relating to a user, such as a user's stool sample analyzed for one or more microorganism strains. In yet another non-limiting example, a performance profile 112 may include a user's toxicity levels, specifying that a user has high levels of cadmium in the user's blood. In yet another non-limiting example, a performance profile 112 may be obtained from sensor data, including any of the sensor data as described above in more detail in reference to FIG. 1. For example, a performance profile 112 may contain information about a user's heart rate while briskly walking, obtained from a sensor. Information relating to performance profile 112 may be stored within user database 120. Information relating to performance profile 112 may be obtained using a questionnaire as described above in more detail in reference to FIG. 1.

With continued reference to FIG. 7, at step 715, computing device 104 determines edible 124 of interest. Edible 124 includes any of the edibles as described above in more detail in reference to FIG. 1. Edible 124 may include a meal option, such as a dinner option containing grilled salmon with asparagus and rice pilaf. In yet another non-limiting example, an edible may include a beverage option, such as an iced latte made with coconut milk. Computing device 104 determines an edible of interest, using a user's dietary habits. Information relating to edibles 124 and/or a user's dietary habits may be stored within edible database 128. For example, a user's dietary habits may indicate that a user likes red meat and chicken and dislikes all seafood. In such an instance, computing device 104 may identify edible 124 of interest such as a lunch option that contains a bun less hamburger served with a side of sweet potato fries and a salad. One or more edibles 124 of interest, may be displayed to a user within display interface 108, whereby a user may select one or more edibles 124 of interest that look appealing to a user, and/or that a user may be interested in ordering from an edible provider, such as a meal maker.

With continued reference to FIG. 7, computing device 104 may determine edible 124 of interest based on a user's geolocation. Computing device 104 receives an element of user geolocation data. Geolocation data includes any of the geolocation data as described above in more detail in reference to FIG. 1. Geolocation data may be obtained from a GPS located within user client device 116, and/or from a user input describing a user's location. Computing device 104 identifies a plurality of edibles as a function of the element of user geolocation data. For example, computing device 104 may generate a query to locate edibles available for purchase in Omaha, Nebraska, based on a user's geolocation in Omaha, Nebraska. Computing device 104 displays a plurality of edibles 124 within display interface 108 and receives a user selection containing edible 124 of interest.

With continued reference to FIG. 7, at step 720, computing device 104 receives nourishment information 132 relating to edible 124 of interest. Nourishment information 132 includes any of the nourishment information 132 as described above in more detail in reference to FIG. 1. Nourishment information 132 may be stored within edible database 128 and may be received from third party device 136 operated by a third-party, such as an edible provider, meal-maker, scientist, nutritionist, dietician, medical professional, and the like. Nourishment information 132 may contain any of the nourishment information 132 as described above in more detail in reference to FIG. 1. Nourishment information 132 may contain a caloric input, such as a total number of calories contained within edible 124. For example, nourishment information 132 may specify that a small serving of shrimp scampi over linguini has a total of 840 calories in the entire serving. Nourishment information 132 may contain a nutrient input, describing the total quantities of one or more nutrients contained within edible 124. For example, a nutrient input for an edible containing a medium portion of vegan macaroni and cheese may specify that the vegan macaroni and cheese contains 14 grams of fat, 1 gram of saturated fat, 2 grams of polyunsaturated fat, 4 grams of monounsaturated fat, 200 milligrams of cholesterol, 50 grams of carbohydrates, 8 grams of fiber, and 14 grams of protein. Computing device 104 may determine nourishment information 132 by generating a dietary classifier, which may be implemented as any of the classifiers as described above in more detail in reference to FIG. 1. Dietary classifier uses edible 124 of interest as an input, and outputs a dietary label using a classification process. Classification process includes any of the classification processes as described above in more detail in reference to FIG. 1.

With continued reference to FIG. 7, at step 725 computing device 104 generates a score machine-learning process 140. Score machine-learning process 140 may be implemented as any of the machine learning processes as described above in more detail in reference to FIGS. 1-5. Generating score machine-learning process 140 includes training score machine-learning process 140 using edible training data 144. Edible training data 144 may be implemented as any of the training data as described above in more detail in reference to FIG. 1. Edible training data 144 includes a plurality of data entries containing a performance profile and nourishment information relating to an edible correlated to an edible score. Generating score machine-learning process 140 includes calculating the score machine-learning process 140, where the score machine-learning process 140 uses a performance profile 112 and nourishment information 132 as an input, and outputs an edible score 148.

With continued reference to FIG. 7, at step 730, computing device 104 displays edible score 148 within display interface 108. Displaying edible score 148 includes calculating a first edible score 148 for a first edible 124 and calculating a second edible score 148 for a second edible 124. Computing device 104 charts first edible score 148 as a function of second edible score 148 and displays the chart within display interface 108. This may be performed for example, as described above in more detail in reference to FIG. 6.

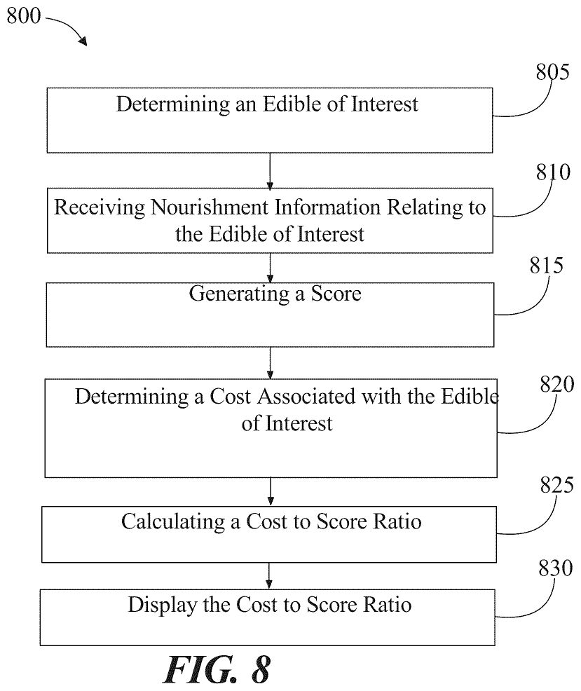

Referring now to FIG. 8, an exemplary embodiment of a method 800 of calculating a score for an edible in a display interface is illustrated. At step 805, a computing device determines an edible of interest; this may be implemented, without limitation, as described above in reference to FIGS. 1-7. At step 810, computing device receives nourishment information relating to the edible of interest to a user; this may be implemented, without limitation, as described above in reference to FIGS. 1-7.

At step 815, and still referring to FIG. 8, computing device generates a score; this may be implemented, without limitation, as described above in reference to FIGS. 1-7. Generating score includes training a score machine-learning process using edible training data, wherein edible training data contains a plurality of data entries, each data entry containing a performance profile and nourishment information and a correlated score data and generating the score as a function of the score machine-learning process, wherein the score machine-learning process uses the performance profile and the nourishment information relating to the edible of interest as an input, and outputs the score.

At step 820, and continuing to refer to FIG. 8, computing device determines a cost associated with edible. Cost may include, without limitation any cost and/or any output of any loss function as described in U.S. Nonprovisional application Ser. No. 16/888,303, filed on May 29, 2020, and entitled “METHODS AND SYSTEMS OF ALIMENTARY PROVISIONING,” the entirety of which is incorporated herein by reference.

At step 825, computing device calculates a cost to score ratio as a function of cost and score; this may be implemented, without limitation, as described above in reference to FIGS. 1-7. At step 830, computing device displays cost to score ratio within a display interface; this may be implemented, without limitation, as described above in reference to FIGS. 1-7.

Still referring to FIG. 8, method 800 may include calculating a performance profile associated with the user. Performance profile may include a biological extraction. Performance profile may include a questionnaire. Edible of interest may be determined as a function of a user dietary habit. Method 800 may include receiving an element of user geolocation data, identifying a plurality of edibles as a function of the element of user geolocation data, displaying the plurality of edibles within the display interface, and receiving a user selection containing the edible of interest. Nourishment information may include a caloric input. Nourishment information may include a nutrient input. Method 800 may include generating a dietary classifier, wherein the dietary classifier uses the edible of interest as an input, and outputs a dietary label using a classification process. Method 800 may include calculating a cost to score ratio for a first edible, calculating a second cost to score ratio for a second edible, charting the first cost to score ratio as a function of the second cost to score ratio, and displaying the first cost to score ratio as a function of the second cost to score ratio within the display interface.

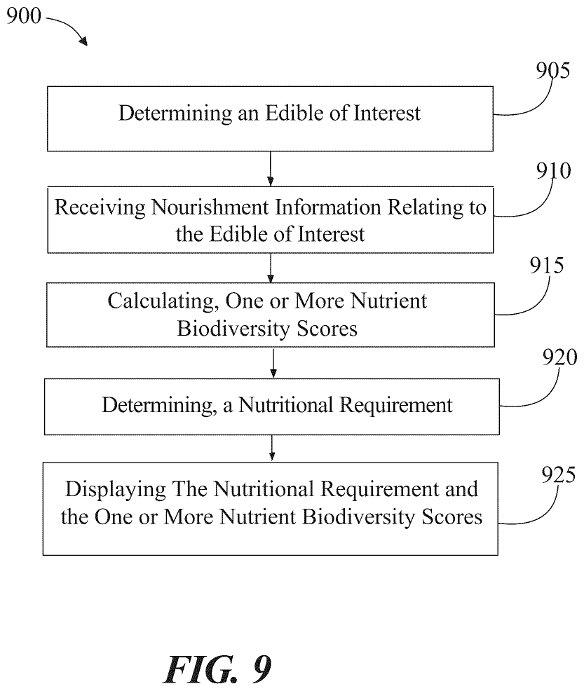

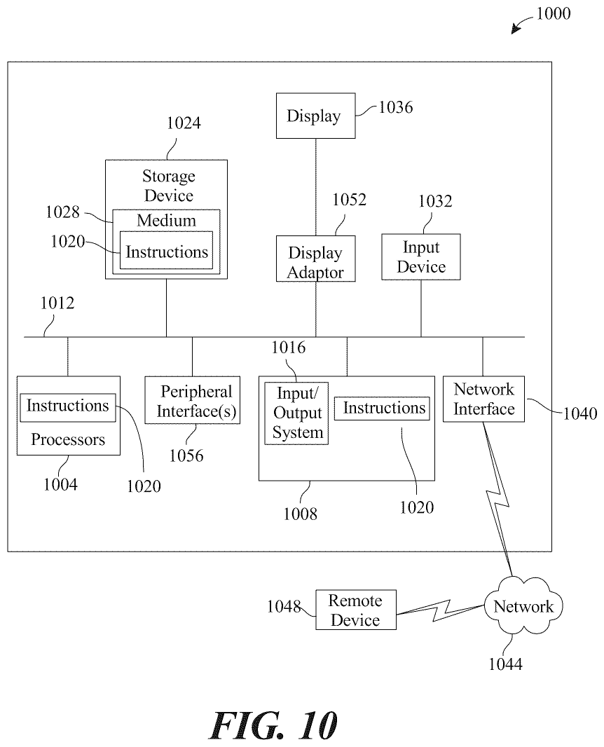

Referring now to FIG. 9, a method 900 for calculating a score for an edible in a display interface is described. In one or more embodiments, at step 905, method 900 includes determining, using a computing device, an edible of interest relating to a user. This may be implemented with reference to FIGS. 1-9 and without limitation.

With continued reference to FIG. 9 at step 910 method 900 includes receiving, using the computing device, nourishment information relating to the edible of interest to the user, wherein the nourishment information includes a plurality of ingredients. This may be implemented with reference to FIGS. 1-9 and without limitation.

With continued reference to FIG. 9, at step 915, method 900 includes calculating, using the computing device, one or more nutrient biodiversity scores as a function of the nourishment information including evaluating each ingredient of the plurality of ingredients, wherein evaluating each ingredient includes extracting at least a nutrient from each ingredient of the plurality of ingredients and calculating a nutrient biodiversity score for the at least a nutrient. This may be implemented with reference to FIGS. 1-9 and without limitation.

With continued reference to FIG. 9, at step 920 method 900 further includes determining, using the computing device, a nutritional requirement as a function of at least the nourishment information. With continued reference to FIG. 9, at step 925, method 900 includes displaying, using the computing device, the nutritional requirement and the one or more nutrient biodiversity scores of the edible of interest through a display interface. In one or more embodiments, method 900 includes retrieving, using the computing device, a performance profile comprising a plurality of logged user performance metrics and generating, using the computing device, an edible score of the edible of interest, wherein generating the edible score includes training a score machine-learning process using edible training data, wherein edible training data contains a plurality of data entries, each data entry containing elements of the performance profile and the nourishment information correlated to an edible score and generating the edible score as a function of the score machine-learning process. This may be implemented with reference to FIGS. 1-9 and without limitation.

With continued reference to FIG. 9, in one or more embodiments, the edible of interest includes one or more meal options. In one or more embodiments, wherein determining, using the computing device, the one or more meal option includes selecting a support advisor from a plurality of support advisors and receiving the one or more meal options from the selected support advisor as a function of a plurality of logged user performance metrics. In one or more embodiments, determining, suing the computing device, the edible of interest relating to a user includes retrieving a performance profile comprising a plurality of logged user performance metrics and generating the edible of interest as a function of the plurality of logged user performance metrics. In one or more embodiments, method 900 further includes receiving, by the computing device, a logged performance metric from a plurality of logged performance metrics associated with the edible of interest and displaying, by the computing device, the logged performance metric through a display interface. In one or more embodiments, determining, by the computing device, the edible of interest relating to the user includes determining the edible of interest as a function of a user dietary habit. In one or more embodiments, determining, by the computing device, the edible of interest relating to the user includes retrieving a plurality of performance profiles comprising a plurality of logged performance metrics, and determining the edible of interest as a function of the plurality of logged metrics. In one or more embodiments, method 900 further includes optimizing, by the computing device, the plurality of ingredients as a function of each nutrient biodiversity score. In one or more embodiments, determining, by the computing device, the edible of interest relating to the user comprises receiving an edible of interest from a user client device. This may be implemented with reference to FIGS. 1-9 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Memory 1008 may include various components (e.g., machine-readable media) including, but not limited to, a random access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 13104 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for calculating a score for an edible in a display interface, the system comprising a computing device configured to:
determine an edible of interest relating to a user;
receive nourishment information relating to the edible of interest to the user, wherein the nourishment information comprises a plurality of ingredients;
calculate one or more nutrient biodiversity scores as a function of the nourishment information comprising:
evaluating each ingredient of the plurality of ingredients, wherein evaluating each ingredient includes:
extracting at least a nutrient from each ingredient of the plurality of ingredients; and
calculating a nutrient biodiversity score for the at least a nutrient;
determine a nutritional requirement as a function of at least the nourishment information; and
display the nutritional requirement and the one or more nutrient biodiversity scores of the edible of interest through a display interface.

2. The system of claim 1, wherein the computing device is further configured to:
retrieve a performance profile comprising a plurality of logged user performance metrics; and
generate an edible score of the edible of interest, wherein generating the edible score comprises:
training a score machine-learning process using edible training data, wherein edible training data contains a plurality of data entries, each data entry containing elements of the performance profile and the nourishment information correlated to an edible score; and
generating the edible score as a function of the score machine-learning process.

3. The system of claim 1, wherein the edible of interest comprises one or more meal options.

4. The system of claim 3, wherein determining the one or more meal options comprises:
selecting a support advisor from a plurality of support advisors; and
receiving the one or more meal options from the selected support advisor as a function of a plurality of logged user performance metrics.

5. The system of claim 1, wherein determining the edible of interest relating to a user comprises:
retrieving a performance profile comprising a plurality of logged user performance metrics; and
generating the edible of interest as a function of the plurality of logged user performance metrics.

6. The system of claim 1 wherein the computing device is further configured to:
receive a logged performance metric from a plurality of logged performance metrics associated with the edible of interest; and
display the logged performance metric through a display interface.

7. The system of claim 1, wherein determining the edible of interest relating to the user comprises determining the edible of interest as a function of a user dietary habit.

8. The system of claim 1, wherein determining the edible of interest relating to the user comprises:
retrieving a plurality of performance profiles comprising a plurality of logged performance metrics; and
determining the edible of interest as a function of the plurality of logged performance metrics.

9. The system of claim 1, the computing device further configured to optimize the plurality of ingredients as a function of each nutrient biodiversity score and adjust the plurality of ingredients as a function of the optimization of the plurality of ingredients.

10. The system of claim 1, wherein determining the edible of interest relating to the user comprises receiving an edible of interest from a user client device.

11. A method for calculating a score for an edible in a display interface, the method comprising:
determining, using a computing device, an edible of interest relating to a user;
receiving, using the computing device, nourishment information relating to the edible of interest to the user, wherein the nourishment information comprises a plurality of ingredients;
calculating, using the computing device, one or more nutrient biodiversity scores as a function of the nourishment information comprising:
evaluating each ingredient of the plurality of ingredients, wherein evaluating each ingredient includes:
extracting at least a nutrient from each ingredient of the plurality of ingredients; and
calculating a nutrient biodiversity score for the at least a nutrient;
determining, using the computing device, a nutritional requirement as a function of at least the nourishment information; and
displaying, using the computing device, the nutritional requirement and the one or more nutrient biodiversity scores of the edible of interest through a display interface.

12. The method of claim 11, further comprising:
retrieving, using the computing device, a performance profile comprising a plurality of logged user performance metrics; and
generating, using the computing device, an edible score of the edible of interest, wherein generating the edible score comprises:
training a score machine-learning process using edible training data, wherein edible training data contains a plurality of data entries, each data entry containing elements of the performance profile and the nourishment information correlated to an edible score; and
generating the edible score as a function of the score machine-learning process.

13. The method of claim 11, wherein the edible of interest comprises one or more meal options.

14. The method of claim 13, wherein determining, using the computing device, the one or more meal option comprises:
selecting a support advisor from a plurality of support advisors; and
receiving the one or more meal options from the selected support advisor as a function of a plurality of logged user performance metrics.

15. The method of claim 11, wherein determining, suing the computing device, the edible of interest relating to a user comprises:
retrieving a performance profile comprising a plurality of logged user performance metrics; and
generating the edible of interest as a function of the plurality of logged user performance metrics.

16. The method of claim 11, further comprising:
receiving, by the computing device, a logged performance metric from a plurality of logged performance metrics associated with the edible of interest; and
displaying, by the computing device, the logged performance metric through a display interface.

17. The method of claim 11, wherein determining, by the computing device, the edible of interest relating to the user comprises determining the edible of interest as a function of a user dietary habit.

18. The method of claim 11, wherein determining, by the computing device, the edible of interest relating to the user comprises:
retrieving a plurality of performance profiles comprising a plurality of logged performance metrics; and
determining the edible of interest as a function of the plurality of logged performance metrics.

19. The method of claim 11, further comprising:
optimizing, by the computing device, the plurality of ingredients as a function of each nutrient biodiversity score;
and adjusting, by the computing device, the plurality of ingredients as a function of the optimization of the plurality of ingredients.

20. The method of claim 11, wherein determining, by the computing device, the edible of interest relating to the user comprises receiving an edible of interest from a user client device.
